(19) United States
(12) Patent Application Publication
(10) Pub. No.: US 2008/0112984 A1
(43) Pub. Date: May 15, 2008

(54) Novel Hydrogels Based On Polyvinyl Alcohols And Polyvinyl Alcohol Copolymers

(75) Inventors: Jorg Schulte, Frankfurt Am Main (DE); Tim Dickner, Frankfurt Am Main (DE); Monika Bruckmann, Frankfurt Am Main (DE); Jorg Schottek, Frankfurt (DE); Uwe Blank, Wiesbaden (DE)

Correspondence Address: CONNOLLY BOVE LODGE & HUTZ, LLP, P O BOX 2207, WILMINGTON, DE, 19899, US

(73) Assignee: Celanese Venures GmbH, Frankfurt am Main (DE)

(21) Appl. No.: 11/587,102

(22) PCT Filed: Apr. 22, 2005
(86) PCT No.: PCT/EP05/04348

§ 371 Date: Aug. 17, 2007

(30) Foreign Application Priority Data
Apr. 22, 2004 (DE) 10 2004 019 504.8

(51) Int. Cl. A61K 47/32; C08F 16/06; A61K 8/81; G02C 7/04; C09K 8/24; C08L 29/04

(52) U.S. Cl. 424/400; 525/56; 526/319; 525/57; 514/772.4; 507/221; 351/160 H

(57) ABSTRACT

The invention relates to hydrogels containing polyvinyl alcohols and/or polyvinyl alcohol copolymers having special geometries, methods for the production of said hydrogels, and the use of the hydrogels as materials in the biomedical and pharmaceutical field, for producing contact lenses, for the controlled release of active substances, as a carrier material for inclusion-immobilized biocatalysts, a carrier material for transition metal catalysts, a material for producing reactive membranes, an additive to drilling fluid, a displacing agent in oil recovery, a cement additive and an additive to highly viscous liquids such as crude oil to improve the flowability and increase the speed of conveyance, and a component of cosmetic products.

The present invention relates to hydrogels containing polyvinyl alcohols and/or polyvinyl alcohol copolymers, to methods for producing the hydrogels, and to the use of the hydrogels as materials in the biomedical and pharmaceutical field, for producing contact lenses, for the controlled release of active substances, as a carrier material for inclusion-immobilized biocatalysts, as a carrier material for transition metal catalysts, as a material for producing reactive membranes, as an additive to drilling fluids, as a displacing agent in oil recovery, as an additive for cement and as an additive to highly viscous fluids such as crude oil to improve the flowability and increase the speed of conveyance, and as a component of cosmetic products.

Hydrogels are water-containing gels based on hydrophilic polymers, which are present in the form of three-dimensional networks. As hydrogels, these polymers are insoluble in water but swell up to an equilibrium volume while largely retaining their shape. The network formation takes place predominantly via chemical bonding of the individual polymer chains, but is also possible in a physical manner by means of electrostatic, hydrophobic or dipole/dipole interactions between individual segments of the polymer chains. By selecting the monomers used to build the polymers, the type of crosslinking and the crosslinking density, it is possible to set desired properties of the hydrogels in a targeted manner. The necessary hydrophilicity of the polymers is provided inter alia by hydroxyl, carboxylate, sulphonate or amide groups. Synthetic hydrogels are based inter alia on poly(meth)-acrylic acids, poly(meth)acrylates, polyvinylpyrrolidone or polyvinyl alcohol. Hydrogels are in general very compatible with living tissues (source: Römpp Lexikon Chemie—Version 2.0, Stuttgart/New York: Georg Thieme Verlag 1999).

Hydrogels based on polyvinyl alcohol are known (cf. US2003008396A1, US2001029399A1, US0006231605B1, US0005981826A, WO2001044307A3, WO1998050017A1, Hassan, Christie M.; Peppas, Nikolaos, A.; Advances in Polymer Science (2000), 153 (Biopolymers, PVA Hydrogels Anionic Polymerisation Nanocomposites), 37-65, Peppas, Nikolaos A., Hydrogels Med. Pharm. (1987), 2, 1-48, and the literature cited therein). Hydrogels based on polyvinyl alcohol can be produced by various methods, for example, by repeated freezing and thawing of an aqueous solution of polyvinyl alcohol, by the action of ionizing radiation (UV light, gamma radiation) on an aqueous solution of polyvinyl alcohol, or by reacting polyvinyl alcohol with crosslinking reagents such as glutaraldehyde, acetaldehyde, formaldehyde, maleic acid, oxalic acid, alginic acid, dimethylurea, glyoxal, hydrochloric acid, polyacrolein, diisocyanates and/or divinyl sulphate.

Hydrogels based on polyvinyl alcohols are usually produced from conventional, linear polyvinyl alcohol. In this case, a turbidity of the hydrogel due to crystalline regions occurs, which means that the hydrogels based on conventional polyvinyl alcohol are only suitable to a limited extent for optical applications, such as contact lenses for example. Moreover, hydrogels based on conventional polyvinyl alcohol are not given their mechanical properties until the hydrogel is crosslinked.

Surprisingly, it has now been found that hydrogels based on polyvinyl alcohols with special geometries, such as star-shaped geometries for example, as described in DE10343607 (Schulte et al./Celanese Ventures GmbH) and DE10356574 (Bruckmann et al./Celanese Ventures GmbH), give rise to a lower degree of microcrystallinity in the hydrogel, which leads to improved optical properties, and have improved mechanical properties. Furthermore, the special geometry of the polyvinyl alcohols, as a result of crosslinking the polymers prior to formation of the hydrogel, leads to reduced attrition.

The present invention therefore relates to hydrogels containing at least one polyvinyl alcohol star polymer.

Within the context of the present invention, hydrogels containing at least one polyvinyl alcohol star polymer are understood to mean both hydrogels in which exclusively one or more polyvinyl alcohol star polymers are used as polymer, and mixtures of polyvinyl alcohol star polymers with other polymers. Particular preference is given here to mixtures of polyvinyl alcohol star polymers with conventional polyvinyl alcohol in a ratio of 99:1 to 1:99. Particular preference is also given to mixtures of polyvinyl alcohol star polymers with polyacrylic acids, polymethacrylic acids, polyacrylates, polymethacrylates and polyvinylpyrrolidone in a ratio of 99:1 to 1:99.

In addition to the aforementioned polyvinyl alcohol star polymers, the hydrogels according to the invention also contain water, as the name suggests.

Polyvinyl alcohols with special geometries can be produced by various methods, on the one hand via a transition-metal-catalyzed production of polyvinyl acetate, as described in DE10238659, followed by saponification, as described in DE10343607, or via a copolymerization of vinyl acetate with crosslinking comonomers, followed by saponification, as described in DE10356574.

Hereinbelow, the polyvinyl alcohols and polyvinyl alcohol copolymers described in DE10343607 and DE10356574 will be referred to as polyvinyl alcohol star polymers.

Polyvinyl alcohol star polymers according to DE10343607 and DE10356574 are understood to mean compounds of the formula I, II or III

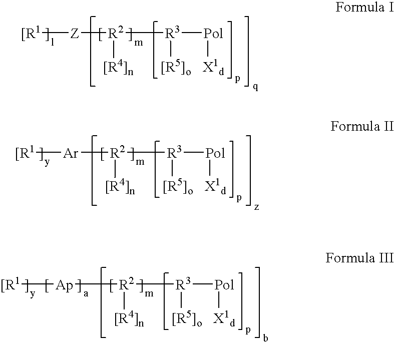

in which
Pol represents a polymer based on a polyvinyl alcohol, particularly preferably a homopolymer or copolymer based on polyvinyl alcohol, very particularly preferably polyvinyl alcohol, polyvinyl alcohol/polyvinyl acetate copolymer, polyvinyl alcohol/polyethylene copolymer, polyvinyl alcohol/polyvinyl chloride copolymer or polyvinyl alcohol/polyacrylic acid methyl ester copolymer, and
Z represents a central atom and is an atom from the 13th to 16th group of the periodic table of the elements, preferably carbon, silicon, nitrogen, phosphorus, oxygen or sulphur, particularly preferably carbon or silicon, and
X¹ is in each case identical or different and is a halogen atom, preferably fluorine, chlorine, bromine or iodine, particularly preferably chlorine, bromine or iodine, and
R¹ is identical or different and is hydrogen or a C₁-C₂₀ carbon-containing group, and
R² is identical or different and is a bridging C₁-C₂₀ carbon-containing group between the central atom Z and the initiating unit [R³—X¹] or is silicon or oxygen, and
R³ is identical or different and is carbon or silicon, and
R⁴ is identical or different and is a hydrogen atom or a C₁-C₂₀ carbon-containing group, and
R⁵ is identical or different and is hydrogen or a C₁-C₂₀ carbon-containing group,
l is a natural integer and is zero, 1, 2 or 3, and
m is in each case identical or different and is a natural integer and is zero, 1, 2, 3, 4 or 5, and
n is in each case identical or different and is a natural integer and is zero, 1, 2, 3, 4, 5, 6, 7, 8, 9, 10, 11, 12, 13, 14, 15, 16, 17, 18, 19 or 20, and
o is in each case identical or different and is 1 or 2, and
p is in each case identical or different and is a natural integer and is 1, 2, 3, 4 or 5, and
q is a natural integer and is 2, 3 or 4, and
Ar is an aromatic skeleton having at least four carbon atoms, in which one or more C atoms may be replaced by boron, nitrogen or phosphorus, and wherein preferred aromatic or heteroaromatic skeletons derive from benzene, biphenyl, naphthalene, anthracene, phenanthrene, triphenylene, quinoline, pyridine, bipyridine, pyridazine, pyrimidine, pyrazine, triazine, benzopyrrole, benzotriazole, benzopyridine, benzopyrazidine, benzopyrimidine, benzopyrazine, benzotriazine, indolizine, quinolizine, carbazole, acridine, phenazine, benzoquinoline, phenoxazine, which may optionally also be substituted, and
y is a natural integer and is zero, 1, 2, 3, 4, 5, 6, 7, 8, 9, 10, 11, 12, 13, 14, 15, 16, 17, 18, 19 or 20, and
z is a natural integer and is 2, 3, 4, 5, 6, 7, 8, 9 or 10,
Ap is a cyclic, non-aromatic skeleton having at least three carbon atoms, which may also contain heteroatoms such as nitrogen, boron, phosphorus, oxygen or sulphur, wherein preferred aliphatic skeletons may be derived from the cycloalkyl group, such as for example cyclopropyl, cyclobutyl, cyclopentyl, cyclohexyl, cycloheptyl, cyclooctyl, cyclononyl, or from the cycloheteroalkyl group, such as for example aziridine, azetidine, pyrrolidine, piperidine, azepane, azocane, 1,3,5-triazinane, 1,3,5-trioxane, oxetane, furan, dihydrofuran, tetrahydrofuran, pyran, dihydropyran, tetrahydropyran, oxepane, oxocane, or from the saccharide group, such as for example alpha-glucose, beta-glucose, and
a is a natural integer and is zero, 1, 2, 3, 4, 5, 6, 7, 8, 9, 10, 11, 12, 13, 14, 15, 16, 17, 18, 19 or 20, and
b is a natural integer and is 2, 3, 4, 5, 6, 7, 8, 9 or 10, and
c is a natural integer and is zero, 1, 2, 3, 4, 5, 6, 7, 8, 9, 10, 11, 12, 13, 14, 15, 16, 17, 18, 19 or 20,
d may be identical or different and is zero or one.

Polyvinyl alcohol copolymers based on polyvinyl ester copolymers are obtainable by a method comprising the steps:
A) radical solution or mass polymerization of vinyl esters in the presence of a radical generator and optionally in the presence of a radical scavenger,
B) addition of a polyalkenyl compound capable of crosslinking,
C) optionally, work-up and isolation of the polyvinyl ester copolymers formed,
D) saponification of the polyvinyl ester/polyvinyl ester polyalkene mixture produced in B) or of the polyvinyl ester/polyalkene copolymers isolated in C) with a base to form the polyvinyl alcohol/polyvinyl alcohol polyalkene mixtures or polyvinyl alcohol polyalkene copolymers and isolation of the products,
   characterized in that the polyalkenyl compounds used in step B) are compounds according to Formula IV:

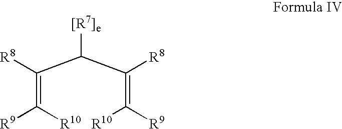

in which:
R⁶ is a C₆-C₂₀ aryl group, a C₅-C₂₀ heteroaryl group, a C₄-C₂₀ cycloalkyl group, a C₄-C₂₀ heterocycloalkyl group or a C₁-C₂₀ alkyl group, in which one or more C atoms which are not directly adjacent to one another may be replaced by an element of the 5th or 6th group of the elements, preferably nitrogen, phosphorus, oxygen or sulphur, particularly preferably nitrogen or oxygen, and
R⁷ is identical or different and is hydrogen, oxygen, sulphur or a hydroxyl group, a carbamoyl group, an amino group, a carboxy group, a C₁-C₂₀ alkylcarbonyl group, a C₁-C₂₀ alkyloxy group, a C₆-C₂₀ aryloxy group, an imino group, a C₁-C₂₀ alkylimino group, a C₆-C₂₀ alkylimino group, a cyano group, a β1-C₂₀ alkyl group, a C₆-C₂₀ aryl group, a C₅-C₂₀ heteroaryl group, a C₄-C₂₀ cycloalkyl group, a C₄-C₂₀ heterocycloalkyl, a C₇-C₂₀ alkylaryl group, a C₇-C₃₀ arylalkyl group, a C₂-C₂₀ alkenyl group, a C₂-C₂₀ α-oxyalkenyl, a halogen-containing C₁-C₂₀ alkyl group, a C₁-C₂₀ aryl group, a C₇-C₂₀ alkylaryl group, a C₇-C₃₀ arylalkyl group or a C₂-C₂₀ alkenyl group, and
R⁸, R⁹, R¹⁰ are identical or different and are hydrogen or a C₁-C₂₀ carbon-containing group, and
e is a natural integer from 0 to 40.

Within the context of the present invention, a C₁-C₂₀ carbon-containing group will be understood to mean preferably the radicals C₁-C₂₀ alkyl, particularly preferably methyl, ethyl, n-propyl, i-propyl, n-butyl, i-butyl, s-butyl, t-butyl, n-pentyl, s-pentyl, cyclopentyl, n-hexyl, cyclohexyl, n-octyl or cyclooctyl, C₁-C₂₀ alkenyl, particularly preferably ethenyl, propenyl, butenyl, pentenyl, cyclopentenyl, hexenyl, cyclohexenyl, octenyl or cyclooctenyl, C₁-C₂₀ alkynyl, particularly preferably ethynyl, propynyl, butynyl, pentynyl, hexynyl or octynyl, C₆-C₂₀ aryl, particularly preferably phenyl, biphenyl, naphthyl or anthracenyl, C₁-C₂₀ fluoroalkyl, particularly preferably trifluoromethyl, pentafluoroethyl or 2,2,2-trifluoroethyl, 06-020 aryl, particularly preferably phenyl, biphenyl, naphthyl, anthracenyl, triphenylenyl, [1,1′;3′,1″]-terphenyl-2′-yl, binaphthyl or phenanthrenyl, C₆-C₂₀ fluoroaryl, particularly preferably tetrafluorophenyl or heptafluoronaphthyl, C₁-C₂₀ alkoxy, particularly preferably methoxy, ethoxy, n-propoxy, i-propoxy, n-butoxy, i-butoxy, s-butoxy or t-butoxy, C₆-C₂₀ aryloxy, particularly preferably phenoxy, naphthoxy, biphenyloxy, anthracenyloxy, phenanthrenyloxy, C₇-C₂₀ arylalkyl, particularly preferably o-tolyl, m-tolyl, p-tolyl, 2,6-dimethylphenyl, 2,6-diethylphenyl, 2,6-di-i-propylphenyl, 2,6-di-t-butylphenyl, o-t-butylphenyl, m-t-butylphenyl, p-t-butylphenyl, C₇-C₂₀ alkylaryl, particularly preferably benzyl, ethylphenyl, propylphenyl, diphenylmethyl, triphenylmethyl or naphthalenylmethyl, C₇-C₂₀ aryloxyalkyl, particularly preferably o-methoxyphenyl, m-phenoxymethyl, p-phenoxymethyl, C₁₂-C₂₀ aryloxyaryl, particularly preferably p-phenoxyphenyl, C₅-C₂₀ heteroaryl, particularly preferably 2-pyridyl, 3-pyridyl, 4-pyridyl, quinolinyl, isoquinolinyl, acridinyl, benzoquinolinyl or benzoisoquinolinyl, C₄-C₂₀ heterocycloalkyl, particularly preferably furyl, benzofuryl, 2-pyrrolidinyl, 2-indolyl, 3-indolyl, 2,3-dihydroindolyl, C₈-C₂₀ arylalkenyl, particularly preferably o-vinylphenyl, m-vinylphenyl, p-vinylphenyl, C₈-C₂₀ arylalkynyl, particularly preferably o-ethynylphenyl, m-ethynylphenyl or p-ethynylphenyl, or a C₂-C₂₀ heteroatom-containing group, particularly preferably carbonyl, benzoyl, oxybenzoyl, benzoyloxy, acetyl, acetoxy or nitrile, wherein one or more C₁-C₂₀ carbon-containing groups may form a cyclic system.

Within the context of the present invention, a bridging C₁-C₂₀ carbon-containing group will be understood to mean preferably C₁-C₂₀ alkyl, particularly preferably methylene, ethylene, propylene, butylene, pentylene, cyclopentylene, hexylene or cyclohexylene, C₁-C₂₀ alkenyl, particularly preferably ethenyl, propenyl, butenyl, pentenyl, cyclopentenyl, hexenyl or cyclohexenyl, C₁-C₂₀ alkynyl, particularly preferably ethynyl, propynyl, butynyl, pentynyl or hexynyl, C₁-C₂₀ aryl, particularly preferably o-phenylene, m-phenylene or p-phenylene, or a C₁-C₂₀ heteroatom-containing group, particularly preferably carbonyl, oxycarbonyl, carbonyloxy, carbamoyl or amido.

The present invention also relates to the production and use of the hydrogels according to the invention.

The hydrogels according to the invention, which contain a polyvinyl alcohol star polymer may be produced for example by:
repeated freezing and thawing of an aqueous solution containing a polyvinyl alcohol star polymer and optionally further components,
the action of ionizing radiation (UV light, gamma radiation) on an aqueous solution containing a polyvinyl alcohol star polymer and optionally further components, and
in aqueous solution containing a polyvinyl alcohol star polymer and optionally further components with crosslinking reagents such as, for example, glutaraldehyde, acetaldehyde, formaldehyde, maleic acid, oxalic acid, alginic acid, dimethylurea, glyoxal, hydrochloric acid, polyacrolein, diisocyanates and/or divinyl sulphate, optionally with acid or base catalysis

Further components are understood to mean, inter alia:
conventional polyvinyl alcohol,
other polymers such as, for example, polyacrylic acids, polymethacrylic acids, polyacrylates, polymethacrylates and polyvinylpyrrolidone,
microorganisms,
alginic acid derivatives, such as sodium alginate for example,
salts such as, for example, sodium carbonate, potassium carbonate, calcium carbonate, ammonium carbonate, magnesium carbonate, sodium hydrogen carbonate, potassium hydrogen carbonate, calcium hydrogen carbonate, magnesium hydrogen carbonate, sodium sulphate, potassium sulphate, ammonium sulphate, magnesium sulphate, sodium phosphate, potassium phosphate, ammonium phosphate, magnesium phosphate, sodium chloride, potassium chloride, ammonium chloride, calcium chloride, magnesium chloride and/or aluminium chloride.

Compared to hydrogels based on conventional polymers, the hydrogels according to the invention are characterized by a higher level of clarity (comparison of the haze values: see Examples 3 and 4).

Compared to hydrogels based on conventional polymers, the hydrogels according to the invention are characterized by a higher level of resistance to water, which is advantageous when used as a carrier material for inclusion-immobilized biocatalysts, for example, for wastewater treatment (comparison of the resistance to water: see Examples 7 and 8).

Examples of hydrogels according to the invention, which serve to explain but not limit the invention, include products based on mixtures of:
polyvinyl alcohol star polymer(s) and water,
polyvinyl alcohol star polymer(s), conventional polyvinyl alcohol and water,
polyvinyl alcohol star polymer(s), polyacrylic acid and water,
polyvinyl alcohol star polymer(s), polymethacrylic acids and water,
polyvinyl alcohol star polymer(s), polyacrylates and water,
polyvinyl alcohol star polymer(s), polymethacrylates and water,
polyvinyl alcohol star polymer(s), polyvinylpyrrolidone and water,
polyvinyl alcohol star polymer(s), conventional polyvinyl alcohol, sodium alginate, calcium chloride, formaldehyde and water,
polyvinyl alcohol star polymer(s), conventional polyvinyl alcohol, microorganisms and water,
polyvinyl alcohol star polymer(s), conventional polyvinyl alcohol, calcium chloride and water,
polyvinyl alcohol star polymer(s), conventional polyvinyl alcohol, calcium chloride, microorganisms and water.
   The invention also relates to the use of the hydrogel containing one or more polyvinyl alcohol star polymer(s) as:
materials in the biomedical and pharmaceutical field, for example as artificial tissue or for treating wounds,
as a material for producing contact lenses,
as a material for the controlled release of active substances,
as a carrier material for inclusion-immobilized biocatalysts, for example for wastewater treatment,
as a carrier material for transition metal catalysts, for example palladium catalysts,
as a material for producing reactive membranes,
as an additive to drilling fluids, in particular, due to the degradability, as an additive to flushing fluids for offshore applications,
as a displacing agent in oil recovery,
as an additive for cement,
as an additive to highly viscous fluids such as crude oil to improve the flowability and increase the speed of conveyance,
as a component of cosmetic products.

The invention will be explained by the following examples, which nevertheless do not limit the invention in any way.

### EXAMPLE 1

### Preparation of a Polyvinyl Alcohol Star Polymer According to DE10343607

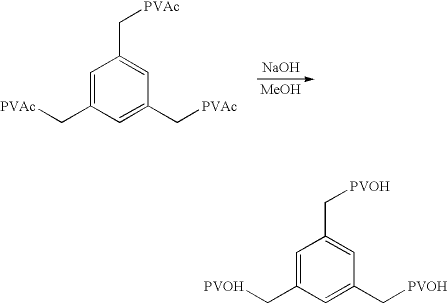

167 ml of a 1% methanolic sodium hydroxide solution in a 1000 ml round-bottomed flask are heated to 50° C. in a water bath. A solution of 50 g of polyvinyl acetate star polymer (produced according to DE10238659) in 333 ml of methanol is added dropwise thereto over a period of 30 min. At the end of the addition, stirring is continued for a further 30 min. The white precipitate is filtered off, washed with methanol until non-alkaline, and dried in vacuo. Yield: 25.0 g. ¹H-NMR (500 MHz, [D₆]-DSMO): δ=6.65 (s, aromatic H), 4.65, 4.46, 3.89, 3.84, 3.31, 1.44-1.33 (4×s, 1×m, PVOH) ppm.

### EXAMPLE 2

### Preparation of a Polyvinyl Alcohol Star Polymer According to DE10356574

132 ml (1.43 mol) of vinyl acetate, 9.0 g (70.2 mmol) of diallylformal, 1.6 ml (7.3 mmol) of tris-(2,2,2-trifluoroethylphosphite and 1.25 g (3.6 mmol) of dibenzoyiperoxide (70%, remainder: H₂0) are placed in 68 ml of toluene. The clear, colourless solution is stirred at 70° C. for 20 h. The volatile components are removed in an oil pump vacuum and the polyvinyl acetate star polymer is taken up in 200 ml of methanol. This solution is added dropwise over 15 min to 400 ml of a 1% NaOH solution in methanol. Stirring is continued for a further 90 min at 50° C. The polyvinyl alcohol is filtered off, washed with methanol until neutral, and dried in the oil pump vacuum. Yield: 14.4 g (22%) ¹H-NMR (500 MHz, [D₆]-DSMO): S=5.92-5.87 (m), 5.24, 5.13 (2×d), 4.66, 4.61, 4.46, 4.22, 4.21, 4.10, 4.09, 4.00, 3.89, 3.84, 3.35, 3.16, 1.43-1.33 (13×m) ppm.

### EXAMPLE 3

### Preparation of a Hydrogel Containing a Polyvinyl Alcohol Star Polymer

25 g of polyvinyl alcohol star polymer (Example 1) are dissolved in 75 ml of dematerialized water at 70° C. The solution is placed in a flat metal dish (20×20 cm), covered with a lid and stored for 12 h at −30° C. Thereafter, the solution is allowed to come to room temperature over 12 h and the freezing/thawing process is repeated a further two times. The hydrogel is obtained in the form of a film having a thickness of 0.25 cm. Turbidity (haze value in percent): 1.3.

### EXAMPLE 4

### Preparation of a Hydrogel Containing a Linear Polyvinyl Alcohol

### Comparative Example

25 g of polyvinyl alcohol (Celvol 103 from Celanese) are dissolved in 75 ml of dematerialized water at 70° C. The solution is placed in a flat metal dish (20×20 cm), covered with a lid and stored for 12 h at −30° C. Thereafter, the solution is allowed to come to room temperature over 12 h and the freezing/thawing process is repeated a further two times. The hydrogel is obtained in the form of a film having a thickness of 0.25 cm. Turbidity (haze value in percent): 4.6.

### EXAMPLE 5

### Preparation of a Hydrogel Containing a Mixture of a Polyvinyl Alcohol Star Polymer with a Linear Polyvinyl Alcohol

2 g of polyvinyl alcohol star polymer (Example 2), 2 g of polyvinyl alcohol (Celvol 103 from Celanese), 0.5 g of alginic acid sodium salt and 0.15 g of sodium hydrogen carbonate are dissolved in 50 ml of water at 70° C. and, after cooling to room temperature, are added dropwise to 250 ml of a 0.1 M calcium chloride solution in large droplets from a syringe without a cannula, with slow stirring, whereby spherical particles having a size of approx. 3 mm are formed. The particles are isolated by means of careful filtration through a glass frit, and are added to a 40° C. solution of 5 g of formaldehyde (37% in water), 50 g of concentrated sulphuric acid and 25 g of sodium sulphate. After one hour, the solidified spherical particles are isolated by filtration and washed with water until neutral. Yield: 43 g of hydrogel as spherical particles.

### EXAMPLE 6

### Preparation of a Hydrogel Containing a Linear Polyvinyl Alcohol

### Comparative Example

4 g of polyvinyl alcohol (Celvol 103 from Celanese), 0.5 g of alginic acid sodium salt and 0.15 g of sodium hydrogen carbonate are dissolved in 50 ml of water at 70° C. and, after cooling to room temperature, are added dropwise to 250 ml of a 0.1 M calcium chloride solution in large droplets from a syringe without a cannula, with slow stirring, whereby spherical particles having a size of approx. 3 mm are formed. The particles are isolated by means of careful filtration through a glass frit, and are added to a 40° C. solution of 5 g of formaldehyde (37% in water), 50 g of concentrated sulphuric acid and 25 g of sodium sulphate. After one hour, the solidified spherical particles are isolated by filtration and washed with water until neutral. Yield: 37 g of hydrogel as spherical particles.

### EXAMPLE 7

### Study of the Attrition of the Hydrogel from Example 5

10 g of hydrogel from Example 5 were placed in a 50 ml one-way syringe, on the bottom of which there is a plastic sieve (mesh width 0.75 mm). The hydrogel was covered with a plastic sieve and the hollow space above the hydrogel was filled with glass wool. The syringe was closed with a rubber stopper, through which a 1 mm Teflon hose passed. Using a pump, tap water was passed through the Teflon hose at a flow rate of 1 ml/min over the hydrogel, over a period of one week. Thereafter, the hydrogel was removed from the syringe and weighed. After one week, 9.2 g of hydrogel from Example 5 were isolated.

### EXAMPLE 8

### Study of the Attrition of the Hydrogel from Example 6

### Comparative Example

10 g of hydrogel from Example 6 were placed in a 50 ml one-way syringe, on the bottom of which there is a plastic sieve (mesh width 0.75 mm). The hydrogel was covered with a plastic sieve and the hollow space above the hydrogel was filled with glass wool. The syringe was closed with a rubber stopper, through which a 1 mm Teflon hose passed. Using a pump, tap water was passed through the Teflon hose at a flow rate of 1 ml/min over the hydrogel, over a period of one week. Thereafter, the hydrogel was removed from the syringe and weighed. After one week, 8.1 g of hydrogel from Example 6 were isolated.

1-6. (canceled)

7. A hydrogel which comprises at least one polyvinyl alcohol star polymer.

8. The hydrogel according to claim 7, which further comprises at least two polyvinyl alcohol star polymers.

9. The hydrogel according to claim 7, which further comprises at least one further polymer wherein at least one further polymer is selected from the group consisting polyvinyl alcohol, polyacrylic acid, polymethacrylic acid, polyacrylate, polymethacrylate and polyvinylpyrrolidone.

10. The hydrogel according to claim 7, wherein the polyvinyl alcohol star polymer is at least one compound of formula I, II or III 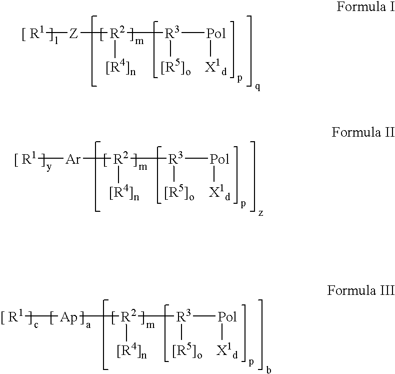
in which
Pol represents a polymer based on a polyvinyl alcohol,
Z represents a central atom and is an atom from the 13th to 16th group of the periodic table of the elements,
X¹ is in each case identical or different and is a halogen atom,
R¹ is in each case identical or different and is hydrogen or a C₁-C₂₀ carbon-containing group,
R² is in each case identical or different and is a bridging C₁-C₂₀ carbon-containing group between the central atom Z and the initiating unit (R³—X¹) or is silicon or oxygen,
R³ is in each case identical or different and is carbon or silicon,
R⁴ is in each case identical or different and is a hydrogen atom or a C₁-C₂₀ carbon-containing group,
R⁵ is in each case identical or different and is hydrogen or a C₁-C₂₀ carbon-containing group
l is a natural integer and is zero, 1, 2 or 3,
m is in each case identical or different and is a natural integer and is zero, 1, 2, 3, 4 or 5,
n is in each case identical or different and is a natural integer and is zero, 1, 2, 3, 4, 5, 6, 7, 8, 9, 10, 11, 12, 13, 14, 15, 16, 17, 18, 19 or 20,
o is in each case identical or different and is 1 or 2,
p is in each case identical or different and is a natural integer and is 1, 2, 3, 4 or 5,
q is a natural integer and is 2, 3 or 4,
Ar is an aromatic skeleton having at least four carbon atoms, in which one or more C atoms is optionally replaced by boron, nitrogen or phosphorus,
y is a natural integer and is zero, 1, 2, 3, 4, 5, 6, 7, 8, 9, 10, 11, 12, 13, 14, 15, 16, 17, 18, 19 or 20,
z is a natural integer and is 2, 3, 4, 5, 6, 7, 8, 9 or 10,
Ap is a cyclic, non-aromatic skeleton having at least three carbon atoms, which optionally contains at least one heteroatom,
a is a natural integer and is zero, 1, 2, 3, 4, 5, 6, 7, 8, 9, 10, 11, 12, 13, 14, 15, 16, 17, 18, 19 or 20,
b is a natural integer and is 2, 3, 4, 5, 6, 7, 8, 9 or 10,
c is a natural integer and is zero, 1, 2, 3, 4, 5, 6, 7, 8, 9, 10, 11, 12, 13, 14, 15, 16, 17, 18, 19 or 20, and
d may be identical or different and is zero or one.

11. The hydrogel according to claim 10, wherein
Pol represents a homopolymer or copolymer based on polyvinyl alcohol,
Z represents a central atom and is carbon, silicon, nitrogen, phosphorus, oxygen or sulphur,
X¹ is in each case identical or different and is fluorine, chlorine, bromine or iodine,
R¹ is in each case identical or different and is hydrogen or a C₁-C₂₀ carbon-containing group,
R² is in each case identical or different and is a bridging C₁-C₂₀ carbon-containing group between the central atom Z and the initiating unit (R³—X¹) or is silicon or oxygen,
R³ is in each case identical or different and is carbon or silicon,
R⁴ is in each case identical or different and is a hydrogen atom or a C₁-C₂₀ carbon-containing group,
R⁵ is in each case identical or different and is hydrogen or a C₁-C₂₀ carbon-containing group
l is a natural integer and is zero, 1, 2 or 3,
m is in each case identical or different and is a natural integer and is zero, 1, 2, 3, 4 or 5,
n is in each case identical or different and is a natural integer and is zero, 1, 2, 3, 4, 5, 6, 7, 8, 9, 10, 11, 12, 13, 14, 15, 16, 17, 18, 19 or 20,
o is in each case identical or different and is 1 or 2,
p is in each case identical or different and is a natural integer and is 1, 2, 3, 4 or 5,
q is a natural integer and is 2, 3 or 4,
Ar is an aromatic skeleton having at least four carbon atoms, in which one or more C atoms is optionally replaced by boron, nitrogen or phosphorus, and wherein said aromatic or heteroaromatic skeleton is derived from benzene, biphenyl, naphthalene, anthracene, phenanthrene, triphenylene, quinoline, pyridine, bipyridine, pyridazine, pyrimidine, pyrazine, triazine, benzopyrrole, benzotriazole, benzopyridine, benzopyrazidine, benzopyrimidine, benzopyrazine, benzotriazine, indolizine, quinolizine, carbazole, acridine, phenazine, benzoquinoline, phenoxazine, which may optionally also be substituted,
y is a natural integer and is zero, 1, 2, 3, 4, 5, 6, 7, 8, 9, 10, 11, 12, 13, 14, 15, 16, 17, 18, 19 or 20,
z is a natural integer and is 2, 3, 4, 5, 6, 7, 8, 9 or 10,
Ap is a cyclic, non-aromatic skeleton having at least three carbon atoms, which optionally contains nitrogen, boron, phosphorus, oxygen or sulphur, wherein said aliphatic skeletons is derived from a cycloalkyl group, a cycloheteroalkyl group, or a saccharide group,
a is a natural integer and is zero, 1, 2, 3, 4, 5, 6, 7, 8, 9, 10, 11, 12, 13, 14, 15, 16, 17, 18, 19 or 20,
b is a natural integer and is 2, 3, 4, 5, 6, 7, 8, 9 or 10,
c is a natural integer and is zero, 1, 2, 3, 4, 5, 6, 7, 8, 9, 10, 11, 12, 13, 14, 15, 16, 17, 18, 19 or 20, and
d may be identical or different and is zero or one.

12. The hydrogel according to claim 10, wherein
Pol represents polyvinyl alcohol, polyvinyl alcohol/polyvinyl acetate copolymer, polyvinyl alcohol/polyethylene copolymer, polyvinyl alcohol/polyvinyl chloride copolymer or polyvinyl alcohol/polyacrylic acid methyl ester copolymer, and
Z represents a central atom and is carbon or silicon,
X¹ is in each case identical or different and is chlorine, bromine or iodine,
R¹ is in each case identical or different and is hydrogen or a C₁-C₂₀ carbon-containing group,
R² is in each case identical or different and is a bridging C₁-C₂₀ carbon-containing group between the central atom Z and the initiating unit (R³—X¹) or is silicon or oxygen,
R³ is in each case identical or different and is carbon or silicon,
R⁴ is in each case identical or different and is a hydrogen atom or a C₁-C₂₀ carbon-containing group,
R⁵ is in each case identical or different and is hydrogen or a C₁-C₂₀ carbon-containing group,
l is a natural integer and is zero, 1, 2 or 3,
m is in each case identical or different and is a natural integer and is zero, 1, 2, 3, 4 or 5,
n is in each case identical or different and is a natural integer and is zero, 1, 2, 3, 4, 5, 6, 7, 8, 9, 10, 11, 12, 13, 14, 15, 16, 17, 18, 19 or 20,
o is in each case identical or different and is 1 or 2,
p is in each case identical or different and is a natural integer and is 1, 2, 3, 4 or 5,
q is a natural integer and is 2, 3 or 4,
Ar is an aromatic skeleton having at least four carbon atoms, in which one or more C atoms is optionally replaced by boron, nitrogen or phosphorus, and wherein said aromatic or heteroaromatic skeleton is derived from benzene, biphenyl, naphthalene, anthracene, phenanthrene, triphenylene, quinoline, pyridine, bipyridine, pyridazine, pyrimidine, pyrazine, triazine, benzopyrrole, benzotriazole, benzopyridine, benzopyrazidine, benzopyrimidine, benzopyrazine, benzotriazine, indolizine, quinolizine, carbazole, acridine, phenazine, benzoquinoline, phenoxazine, which may optionally also be substituted,
y is a natural integer and is zero, 1, 2, 3, 4, 5, 6, 7, 8, 9, 10, 11, 12, 13, 14, 15, 16, 17, 18, 19 or 20,
z is a natural integer and is 2, 3, 4, 5, 6, 7, 8, 9 or 10,
Ap is a cyclic, non-aromatic skeleton having at least three carbon atoms, which optionally contains nitrogen, boron, phosphorus, oxygen or sulphur, wherein said aliphatic skeletons is derived from the cyclopropyl, cyclobutyl, cyclopentyl, cyclohexyl, cycloheptyl, cyclooctyl, cyclononyl, aziridine, azetidine, pyrrolidine, piperidine, azepane, azocane, 1,3,5-triazinane, 1,3,5-trioxane, oxetane, furan, dihydrofuran, tetrahydrofuran, pyran, dihydropyran, tetrahydropyran, oxepane, oxocane, alpha-glucose, or beta-glucose,
a is a natural integer and is zero, 1, 2, 3, 4, 5, 6, 7, 8, 9, 10, 11, 12, 13, 14, 15, 16, 17, 18, 19 or 20,
b is a natural integer and is 2, 3, 4, 5, 6, 7, 8, 9 or 10,
c is a natural integer and is zero, 1, 2, 3, 4, 5, 6, 7, 8, 9, 10, 11, 12, 13, 14, 15, 16, 17, 18, 19 or 20, and
d may be identical or different and is zero or one.

13. The hydrogel according to claim 7, wherein the polyvinyl alcohol star polymer is a polyvinyl alcohol copolymer based on polyvinyl ester copolymers, which are obtainable by a method comprising the steps:
A) radical solution or mass polymerization of vinyl esters in the presence of a radical generator and optionally in the presence of a radical scavenger,
B) adding a polyalkenyl compound capable of crosslinking,
C) optionally, working-up and isolating of the polyvinyl ester copolymers formed,
D) saponifing the polyvinyl ester/polyvinyl ester polyalkene mixture produced B) or of the polyvinyl ester/polyalkene copolymers isolated in C) with a base to form the polyvinyl alcohol/polyvinyl alcohol polyalkene mixtures or polyvinyl alcohol/polyalkene copolymers and isolation of the products,
wherein the polyalkenyl compounds used in step B) are compounds according to Formula IV: 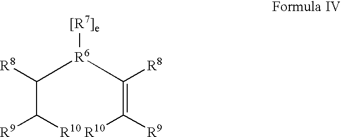
in which:
R⁶ is a C₆-C₂₀ aryl group, a C₅-C₂₀ heteroaryl group, a C₄-C₂₀ cycloalkyl group, a C₄-C₂₀ heterocycloalkyl group or a C₁-C₂₀ alkyl group, in which one or more C atoms which are not directly adjacent to one another may be replaced by an element of the 5th or 6th group of the elements,
R⁷ is identical or different and is hydrogen, oxygen, sulphur or a hydroxyl group, a carbamoyl group, an amino group, a carboxy group, a C₁-C₂₀ alkylcarbonyl group, a C₁-C₂₀ alkyloxy group, a C₆-C₂₀ aryloxy group, an imino group, a C₁-C₂₀ alkylimino group, a C₆-C₂₀ alkylimino group, a cyano group, a C₁-C₂₀ alkyl group, a C₆-C₂₀ aryl group, a C₅-C₂₀ heteroaryl group, a C₄-C₂₀ cycloalkyl group, a C₄-C₂₀ heterocycloalkyl, a C₇-C₂₀ alkylaryl group, a C₇-C₃₀ arylalkyl group, a C₂-C₂₀ alkenyl group, a C₂-C₂₀ α-oxyalkenyl, a halogen-containing C₁-C₂₀ alkyl group, a C₆-C₂₀ aryl group, a C₇-C₂₀ alkylaryl group, a C₇-C₃₀ arylalkyl group or a C₂-C₂₀ alkenyl group,
R⁸, R⁹ and R¹⁰ are identical or different and are hydrogen or a C₁-C₂₀ carbon-containing group, and
e is a natural integer from 0 to 40.

14. The hydrogel according to claim 13, wherein R⁶ is a C₆-C₂₀ aryl group, a C₅-C₂₀ heteroaryl group, a C₄-C₂₀ cycloalkyl group, a C₄-C₂₀ heterocycloalkyl group or a C₁-C₂₀ alkyl group, in which one or more C atoms which are not directly adjacent to one another may be replaced by nitrogen, phosphorus, oxygen or sulphur.

15. A process to produce the hydrogel as claimed in claim 7 which comprises:
A) radical solution or mass polymerization of vinyl esters in the presence of a radical generator and optionally in the presence of a radical scavenger,
B) adding a polyalkenyl compound capable of crosslinking,
C) optionally, working-up and isolating of the polyvinyl ester copolymers formed,
D) saponifing the polyvinyl ester/polyvinyl ester polyalkene mixture produced B) or of the polyvinyl ester/polyalkene copolymers isolated in C) with a base to form the polyvinyl alcohol/polyvinyl alcohol polyalkene mixtures or polyvinyl alcohol/polyalkene copolymers and isolation of the products,
wherein the polyalkenyl compounds used in step B) are compounds according to Formula IV: 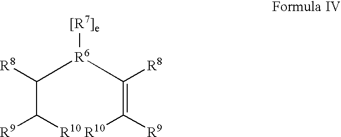
in which:
R⁶ is a C₆-C₂₀ aryl group, a C₅-C₂₀ heteroaryl group, a C₄-C₂₀ cycloalkyl group, a C₄-C₂₀ heterocycloalkyl group or a C₁-C₂₀ alkyl group, in which one or more C atoms which are not directly adjacent to one another may be replaced by an element of the 5th or 6th group of the elements,
R⁷ is identical or different and is hydrogen, oxygen, sulphur or a hydroxyl group, a carbamoyl group, an amino group, a carboxy group, a C₁-C₂₀ alkylcarbonyl group, a C₁-C₂₀ alkyloxy group, a C₆-C₂₀ aryloxy group, an imino group, a C₁-C₂₀ alkylimino group, a C₆-C₂₀ alkylimino group, a cyano group, a C₁-C₂₀ alkyl group, a C₆-C₂₀ aryl group, a C₅-C₂₀ heteroaryl group, a C₄-C₂₀ cycloalkyl group, a C₄-C₂₀ heterocycloalkyl, a C₇-C₂₀ alkylaryl group, a C₇-C₃₀ arylalkyl group, a C₂-C₂₀ alkenyl group, a C₂-C₂₀ α-oxyalkenyl, a halogen-containing C₁-C₂₀ alkyl group, a C₆-C₂₀ aryl group, a C₇-C₂₀ alkylaryl group, a C₇-C₃₀ arylalkyl group or a C₂-C₂₀ alkenyl group, R⁸, R⁹ and R¹⁰ are identical or different and are hydrogen or a C₁-C₂₀ carbon-containing group, and
e is a natural integer from 0 to 40.

16. The method according to claim 15, wherein R⁶ is a C₆-C₂₀ aryl group, a C₅-C₂₀ heteroaryl group, a C₄-C₂₀ cycloalkyl group, a C₄-C₂₀ heterocycloalkyl group or a C₁-C₂₀ alkyl group, in which one or more C atoms which are not directly adjacent to one another may be replaced by nitrogen, phosphorus, oxygen or sulphur.

17. An artificial tissue or for treating wounds which comprises the hydrogel as claimed in claim 7.

18. A contact lens which comprises the hydrogel as claimed in claim 7.

19. A carrier material for the controlled release of active substances which comprises the hydrogel as claimed in claim 7.

20. A carrier material for inclusion-immobilized biocatalysts which comprises the hydrogel as claimed in claim 7.

21. A carrier material for transition metal catalysts which comprises the hydrogel as claimed in claim 7.

22. A material for producing reactive membranes which comprises the hydrogel as claimed in claim 7.

23. An additive to drilling fluids which comprises the hydrogel as claimed in claim 7.

24. A displacing agent in oil recovery which comprises the hydrogel as claimed in claim 7.

25. An additive in building materials or to highly viscous fluids which comprises the hydrogel as claimed in claim 7.

26. A component of cosmetic products which comprises the hydrogel as claimed in claim 7.
